(19) United States
(12) Patent Application Publication
(10) Pub. No.: US 2013/0268091 A1
(43) Pub. Date: Oct. 10, 2013

(54) ADJUSTMENT-FREE MULTI-STAGE PROSTHESIS AIR CYLINDER AND MULTI-STAGE AIR PRESSURE VALVE THEREOF

(71) Applicants: PRO LIMB International Corp., (US); MEDI GMBH & CO., KG, Bayreuth (DE)

(72) Inventor: Hsin-Fa SHEN, Banqiao City (TW)

(73) Assignees: MEDI GMBH & CO., KG, Bayreuth (DE); PRO LIMB INTERRNATIONAL CORP., Shulin City (TW)

(21) Appl. No.: 13/846,026

(22) Filed: Mar. 18, 2013

(30) Foreign Application Priority Data
Apr. 5, 2012 (TW) 101206148

(51) Int. Cl. A61F 2/50

(52) U.S. Cl. CPC A61F 2/50
USPC 623/26

(57) ABSTRACT

An adjustment-free multi-stage prosthesis air cylinder comprises an air cylinder body, a piston, a first check valve and a multi-stage air pressure valve. The inner of the air cylinder body forms an air chamber and a lower air way. One end of the piston is slidably disposed in the air chamber and divides the air chamber into an upper air chamber and a lower air chamber, the other end of the piston extends to the outside; the inner of the piston forms an upper air way which is communicated with the outside and the upper air chamber, while the lower air way is communicated with the lower air chamber. The piston makes a reciprocating movement in the air chamber, and the first check valve is disposed in the piston to optionally connect the upper air way with the lower air way. The multi-stage air pressure valve is disposed in the lower air way. With the arrangement of the multi-stage air pressure valve, the users can easily obtain different walking speeds without changing the air cylinder or setting the operational mode.

### TECHNICAL FIELD

The present creation relates to a cushioning air cylinder, especially to an adjustment-free multi-stage prosthesis air cylinder which has a multi-stage air pressure valve and can be used in prosthesis joint.

### BACKGROUND ART

The air cylinder is a structure which controls air pressure by using the piston's stretch-out and draw-back. It has characteristics of accepting accumulation of pressures and automatically returning after removing of external forces due to the compressibility of air pressure.

The air cylinder is also applied to prosthesis joint and used as medical device providing cushioning function as an alternative of joint. In the common design of the prosthesis joint, the cushioning air cylinder provides cushioning function for certain compression strength. For example, a cushioning air cylinder that corresponds to a slow walking speed is used for the user who slowly walks; while the user wants to conduct a brisk walk or go jogging, it must be replaced by another cushioning air cylinder which adapts to that brisk walk speed. In other words, the user must prepare two or more kinds of cushioning air cylinders in order to be adapted to different walking speeds.

Another relatively new type of cushioning air cylinder has a stepped device to set the level of the cushioning force. The users themselves can adjust the air cylinder setting according to the walking speeds so that the cushioning air cylinder can generate corresponding cushioning capacity.

However, although this type of air cylinder can be individually used to respond to the changes of the environment, the user still needs to actively adjust the device whenever the walking speed is changed, whatever modification is made to the adjustment mode. And, under any condition where a wrong cushioning mode is set, it may not only cause the damage of the cushioning air cylinder, but also more likely to cause the user uncomfortable or even injured for the lack of cushioning capacity.

### SUMMARY OF THE INVENTION

The present creation intends to provide an adjustment-free multi-stage prosthesis air cylinder having a multi-stage air pressure valve, which can be used in prosthesis joint to be automatically adapted to different impact strength under different motion speeds of the joint, so as to improve the environmental adaptability as well as the life span of the prosthesis joint.

The present creation provides a multi-stage air pressure valve 1 which is disposed in the air way comprising a first component, a second component, two sealing rings and an elastic piece with the inner of the first component including a main channel and a first bypass channel. The main channel penetrates through the first component, forms an air inlet on the top surface of the first component and a first air outlet on the bottom surface of the first component. The first bypass channel communicates with the main channel and forms a second air outlet on the side surface of the first component. The second component is optionally sleeved on the first component. A second bypass channel communicating with the first bypass channel is formed between the first component and the second component. The two sealing rings are sleeved on the side surface of the first component to optionally close the second bypass channel, and the second air outlet is located between the two sealing rings. The elastic piece connects the first component with the second component.

The present creation further provides an adjustment-free multi-stage prosthesis air cylinder comprising an air cylinder body, a piston, a first check valve and a multi-stage air pressure valve. An air chamber is formed inside the air cylinder body, and the cylinder body forms a lower air way. One end of the piston is slidably disposed in the air chamber, and the other end of the piston extends out of the air cylinder body. The piston divides the air chamber into an upper air chamber and a lower air chamber. An upper air way is formed inside the piston, having one end connecting to the outside and the other end connecting to the upper air chamber. The lower air chamber communicates with the lower air way, and the piston makes a reciprocating movement in the air chamber. The first check valve is disposed in the piston to optionally connect the upper air chamber with the lower air chamber. The multi-stage air pressure valve that is disposed inside the lower air way comprises a first component, a second component, two sealing rings and an elastic piece. The inner of the first component includes a main channel and a first bypass channel, the main channel penetrates through the first component, forming an air inlet on the top surface of the first component and a first air outlet on the bottom surface of the first component. The first bypass channel communicates with the main channel and forms a second air outlet on the side surface of the first component. The second component is optionally sleeved on the first component, and a second bypass channel communicating with the first bypass channel is formed between the first component and the second component. The two sealing rings are sleeved on the side surface of the first component to optionally close the second bypass channel, and the second air outlet is formed between the two sealing rings; the elastic piece connects the first component with the second component.

The present creation is beneficial in that the adjustment-free multi-stage prosthesis air cylinder of the present creation is suitable for prosthesis joint, mainly suitable for knee prosthesis for legs; when the users are slowly walking (usually with a walking speed less than 2-4 km/h), the gas flow rate is relatively larger, so as to quickly discharge the inside pressure, which enables a longer cushioning stroke for the piston so that the users can feel more comfortable. When the users are walking in a high speed (usually with a walking speed around 4-8 km/h), the gas flow rate of the multi-stage air pressure valve 1 is automatically reduced so that the inner thereof has an instantaneous high pressure, which enables a shorter cushioning stroke for the piston to provide the users with a good operability.

The adjustment-free multi-stage prosthesis air cylinder of the present creation is simple to install, so that the users do not need to adjust the setting of the air pressure valve but only need to assemble the adjustment-free multi-stage prosthesis air cylinder with the prosthesis joint; moreover, since the adjustment-free multi-stage prosthesis air cylinder has a simple structure, lower cost, and longer span lift, it overcomes the defects of the traditional installation that is complex, error-prone and requires frequent maintenance.

The features and the technical contents of the present creation will be further appreciated from the following detailed description and figures which are illustrated for reference and explanation only but not to limit the extent of the scope of the present creation.

### BRIEF DESCRIPTION OF THE FIGURES

FIG. 1A is a cross-section schematic diagram of the first component before compression of the first embodiment of the present creation.

FIG. 1B is a cross-section schematic diagram of the first, component after compression of the first embodiment of the present creation.

FIG. 2 is a cross-section schematic diagram of the first component before compression of the second embodiment of the present creation.

FIG. 2A is a cross-section schematic diagram of the first component during compression of the second embodiment of the present creation.

FIG. 2B is a cross-section schematic diagram of the first component after compression of the second embodiment of the present creation.

FIG. 3 is a cross-section schematic diagram of the third embodiment of the present creation.

FIG. 3A is a schematic diagram showing the airflow when the piston is moving upwards of the third embodiment of the present creation.

FIG. 3B is a schematic diagram showing the airflow when the piston is moving downwards of the third embodiment of the present creation.

FIG. 4 is a cross-section schematic diagram of the third embodiment of the present creation.

### LIST OF REFERENCE NUMERALS

the multi-stage air pressure valve 1, 1′
the first component 11
the top part 111
the protrusion part 112, 112′
the main channel 113
the air inlet 1131
the first air outlet 1132
the first bypass channel 114
the second air outlet 1141
the second component 12
the bottom part 121
the extension part 122
the sealing ring 13
the elastic piece 14
the second bypass channel 15
the air cylinder body 2
the air chamber 21
the upper air chamber 211
the lower air chamber 212
the lower air way 22
the piston 3
the upper air way 31
the first check valve 4
the second check valve 5

The adjustment-free multi-stage prosthesis air cylinder of the present creation uses the multi-stage air pressure valve which is disposed in the air passage to generate cushioning effect and supporting force corresponding to different pressures.

### EMBODIMENT 1

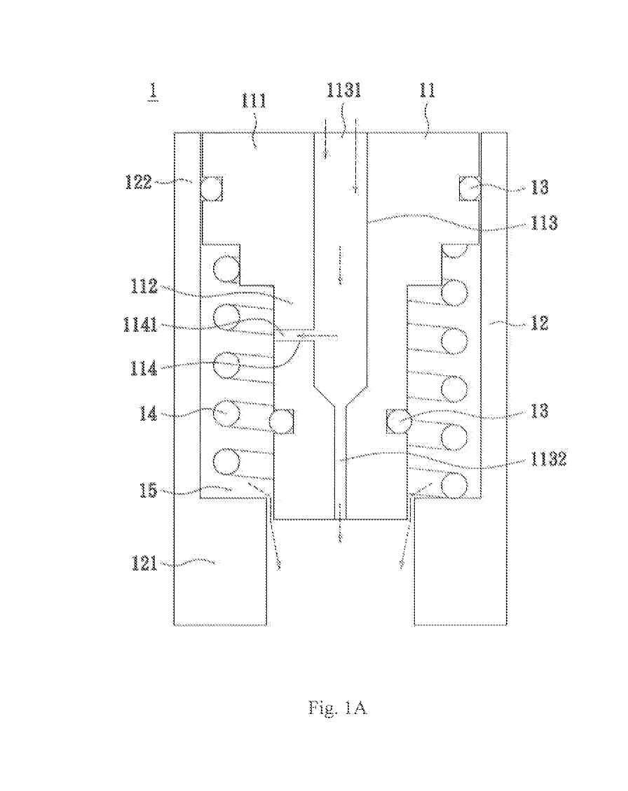

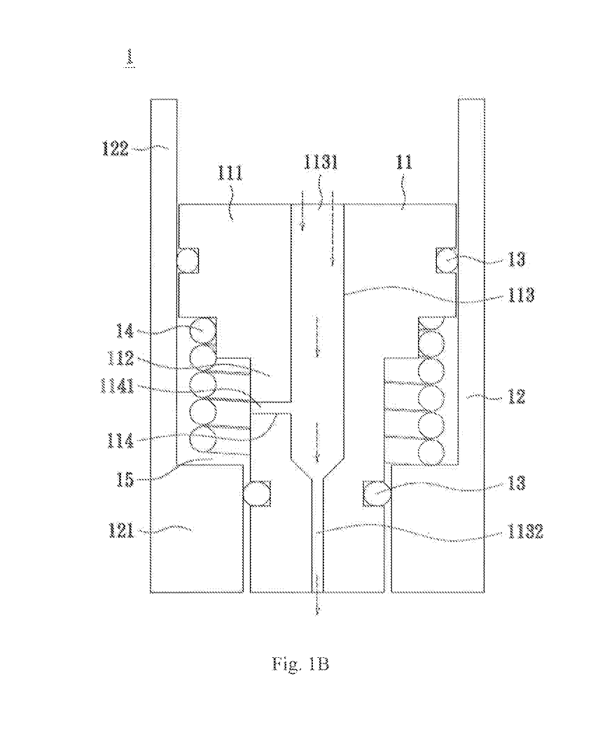

Referring to FIG. 1A, the present embodiment provides a multi-stage air pressure valve 1 which includes a first component 11, a second component 12, an elastic piece 14 and two sealing rings 13. The first component 11 includes two main parts, which are a top part 111 and a protrusion part 112 extending from the top part 111, respectively. A main channel 113 is formed inside the first component 11, penetrating there-through; the main channel 113 forms an air inlet 1131 on the top surface of the first component 11 and a first air outlet 1132 on the bottom surface of the first component 11. The inner of the first component 11 further forms a first bypass channel 114, which has one end connecting to the main channel 113, and the other end penetrating through the first component 11 and forming a second air outlet 1141 on the side surface of the first component 11.

The second component 12 is optionally sleeved on the first component 11, and a second bypass channel 15 is formed between the first component 11 and the second component 12. In details, the second component 12 includes a bottom part 121 and an extension part 122, the extension part 122 radially extends along the direction of the first component 11 and surrounds the top part 111 of the first component 11. The first component 11 slides in the inner of the second component 12, and the protrusion part 112 of the first component 11 optionally slides into the bottom part 121 of the second component 12. Two sealing rings 13 are disposed on the first component 11, in which one is sleeved on the protrusion part 112 and the other one is sleeved on the top part 111; a second air outlet 1141 is located between the two sealing rings 13.

The second bypass channel that is formed between the first component 11 and the second component 12 is optionally sealed by the sealing ring 13. In details, one sealing ring 13 seals the gap between the top part 111 and the extension part 122, and the other sealing ring 13 seals the gap between the protrusion part 112 and the bottom part 121.

An elastic piece 14 is disposed in the sliding direction of the first component 11 and of the second component 12; the elastic piece 14 may be a spring which surrounds the protrusion part 112 of the first component 11, one end of the elastic piece is connecting to the top part 111 of the first component 11, and the other end is connecting to the bottom part 121 of the second component 12.

Referring to FIG. 1A for the operational pattern of the multi-stage air pressure valve 1 of the present creation, when the air enters the air inlet 1131, with the first component 11 not subjected to compressing force, it passes through the main channel 113 and exits from the first outlet 1132. The second bypass channel 15 is in an on-state due to the tap between the protrusion 112 and the bottom 121, so that the air also exits the multi-stage air pressure valve 1 via the second bypass channel 15. Since the main channel 113, the first bypass channel 114 and the second bypass channel 15 are all in an on-state, the multi-stage air pressure valve 1 then has a relatively higher gas flow rate.

Referring to FIG. 18, the first component 11 is subjected to compressing force and slides deeply into the second component 12; under such state, the air enters from the air inlet 1131, passes the main channel 113 and exits from the first air outlet 1132; However, the air in the second side channel 15 can not be discharged because the gap between the protrusion part 112 and the bottom part 121 is sealed. In other words, when the multi-stage air pressure valve 1 is subjected to a certain compressing force which causes the second bypass channel 15 being sealed by the sealing ring 13, the air can only exits the multi-stage air pressure valve 1 via the first air outlet 1132 of the main channel 113, so that the gas flow rate of the multi-stage air pressure valve 1 under compressing force is lower than where there is no compressing force.

It should be supplemented that the sealing ring 13 is disposed between the top part 111 and the extension part 122 for preventing the air of the second bypass channel 15 from couter-flowing to the outside via that gap.

### EMBODIMENT 2

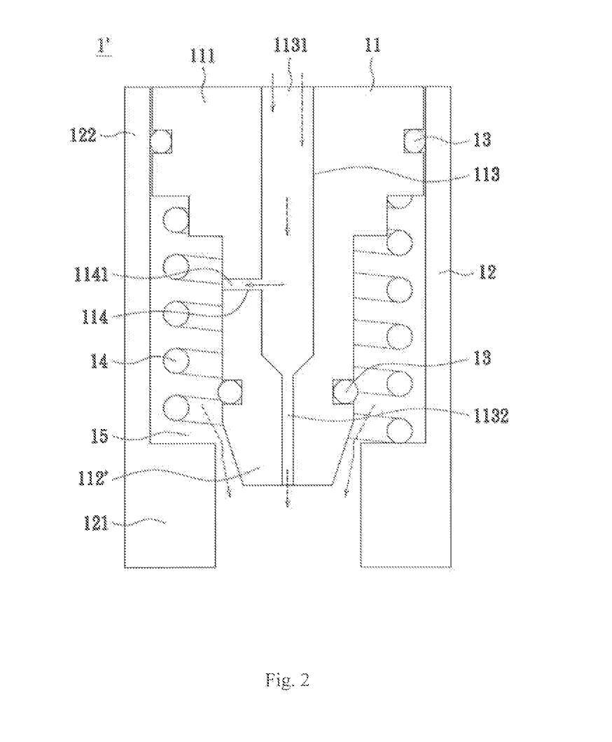

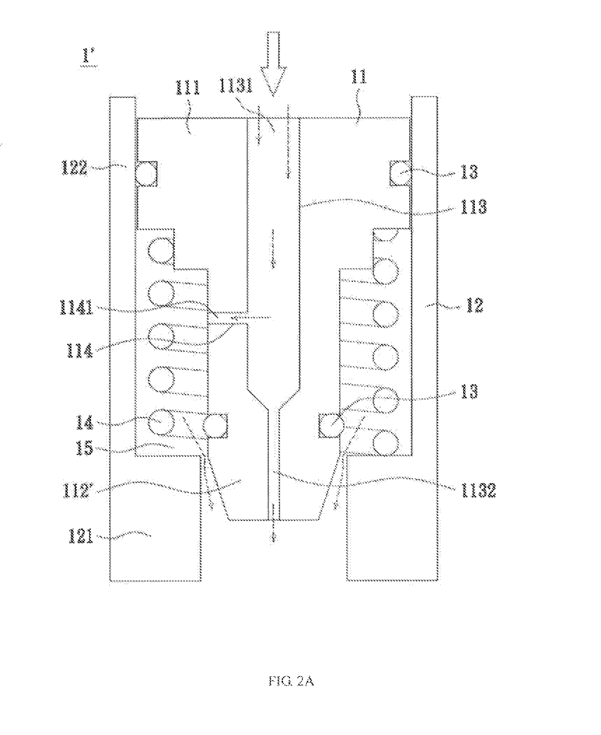

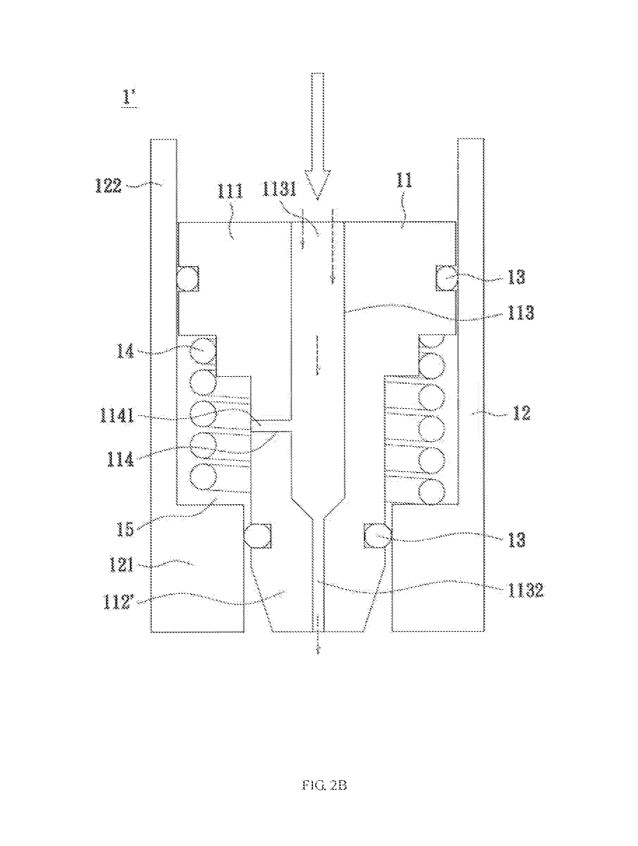

An embodiment of another multi-stage air pressure valve 1 of the present creation is as shown in FIG. 2. The second embodiment is different from the first embodiment in that the protrusion part 112′ of the first component 11 forms into a tapered shape between the second air outlet 1141 and the first air outlet 1132; in other words, the first component 11 and second component 12 form different gaps there-between depending on the different relative positions thereof. The sealing ring 13 is disposed on the starting point of the tapered shape, to optionally seal the gap between the protrusion part 112′ and the bottom part 121.

The operational pattern of the present embodiment is shown in FIG. 2. The first component 11 is subjected to the compressing force and then starts to slide towards the bottom part 121 of the second component 12. At this time, the second bypass channel 15 has a relatively higher gas flow rate due to the greater gap between the protrusion part 112′ and the bottom part 121. As shown in FIG. 2A, the gap between the protrusion part 112′ and the bottom part 121 is gradually narrowed as the protrusion part 112′ of the first component 11 is gradually moved into the bottom part 121 of the second component 12. In other words, the gas flow rate of the second bypass channel 15 is gradually reduced. As shown in FIG. 2B, when the sealing ring 13 seals the gap between the protrusion part 112′ and the bottom part 121, the gas flow rate of the second bypass channel 115 is dropped to zero.

As mentioned above, the gas flow rate of the multi-stage air pressure valve 1 of the present embodiment is provided by the main channel 113, the first bypass channel 114, and the second bypass channel 15. The gas flow rate of the second bypass channel 15 is gradually reduced as the first component 11 is subjected to the compressing force and gradually merged into the second component 12. In other words, the multi-stage air pressure valve 1 of the present embodiment provides different gas flow rates according to different compressing forces to obtain multi-stage adjustment of the air pressure valve.

### EMBODIMENT 3

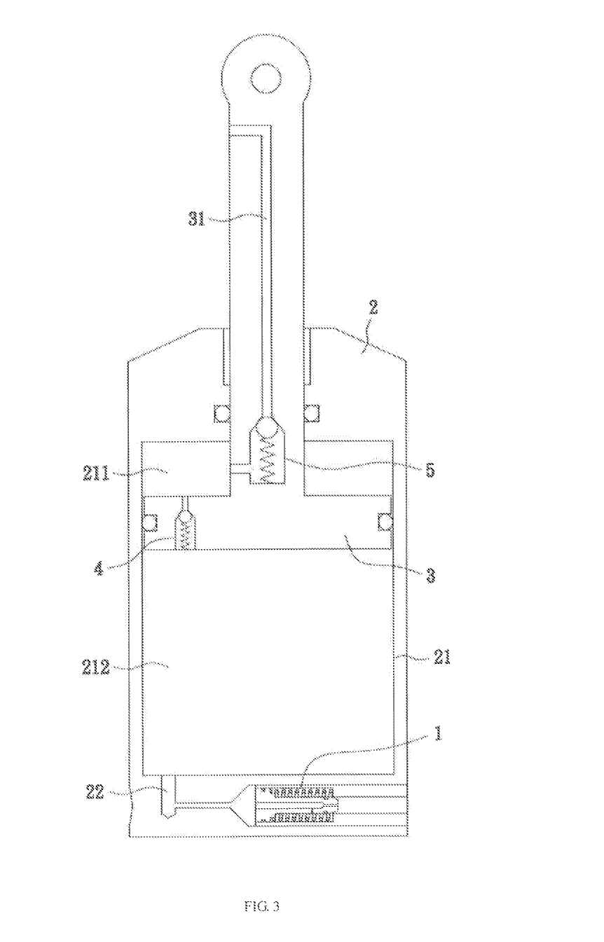

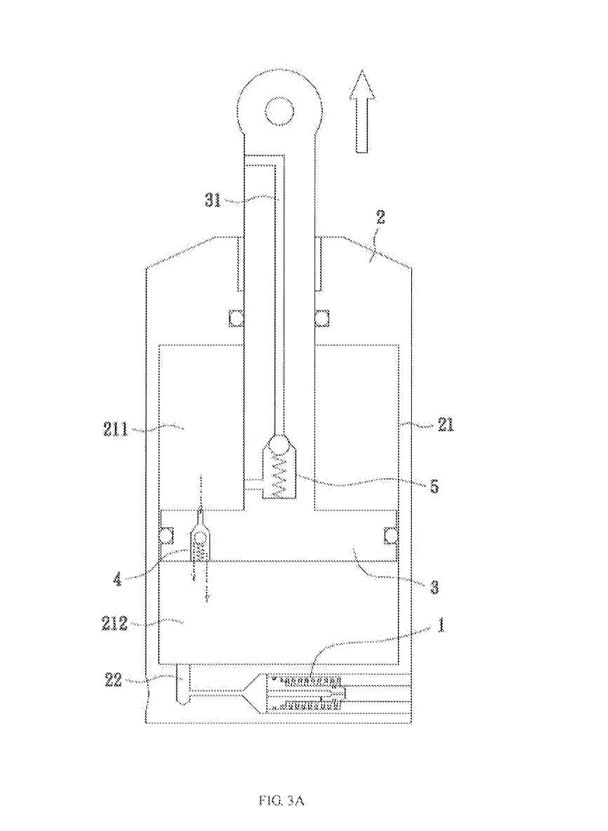

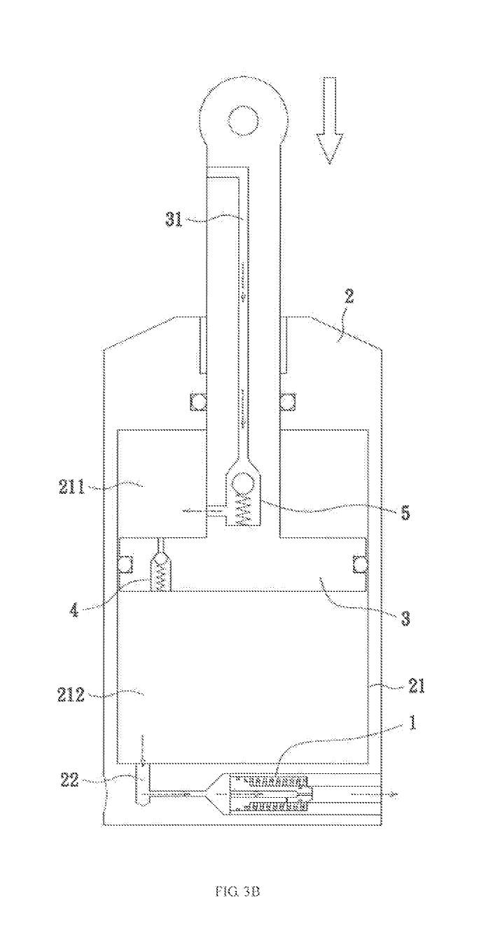

As shown in FIG. 3, the present embodiment provides an adjustment-free multi-stage prosthesis air cylinder having a multi-stage air pressure valve 1 which can be used in prosthesis joint, the adjustment-free multi-stage prosthesis air cylinder comprises an air cylinder body 2, a piston 3, a first check valve 4 and a multi-stage air pressure valve 1.

An air chamber 21 is formed inside the air cylinder body 2, and the piston 3 is disposed inside the air chamber 21.

The piston 3 externally extends out of the cylinder body 2. One end of the piston 3 is slidably disposed inside the air chamber 21, and the other end of the piston 3 extends to the outside of the air cylinder body 2. The piston 3 divides the air chamber 21 into an upper air chamber 211 and a lower air chamber 212, and the piston 3 optionally slides in the air chamber 21 to make a reciprocating movement, so that volumes of the upper air chamber 211 and the lower air chamber 212 are variable.

The first check valve 4 is disposed in the piston 3, having one end connecting to the upper air chamber 211, and the other end connecting to the lower air chamber 212. The air in the upper air chamber 211 can enter the lower air chamber 212 through the first check valve 4, but the air in the lower air chamber 212 can not counter-flow into the upper air chamber 211 due to the stop function of the first check valve 4.

An upper air way 31 is formed inside the piston 3, the two ends of the upper air way 31 are connected to the outside and to the upper air chamber 211, respectively, and a second check valve 5 is disposed inside the upper air way 31. A lower air way 22 is formed inside the air cylinder body 2, and the two ends of the lower air way 22 are connected to the outside and to the lower air chamber 212, respectively.

As shown in FIG. 3A, when the piston 3 moves upwards, the volume of the upper air chamber 211 is gradually reduced and the volume of the lower air chamber 212 is gradually increased. At this moment, the air pressure of the upper air chamber 211 rises, the first check valve 4 is subjected to a pressure difference and opened, while the second check valve 5 is closed to allow the air from the upper air chamber 211 filling the lower air chamber 212. As shown in FIG. 3B and FIG. 1A, when the piston 3 slowly moves downwards, the volume of the upper air chamber 211 is increased and the volume of the lower air chamber 212 is reduced. At this moment, the air pressure of the upper air chamber 211 drops, the second check valve 5 is opened to allow the external air filling the upper air chamber 211, while the first check valve 4 is closed so that the air in the lower air chamber 212 is compressed by the piston 3, flows into the lower air way 22 and is discharged from the air cylinder body 2 through the main channel 113, the first bypass channel 114 and the second bypass channel 115 of the multi-stage air pressure valve 1. As shown in FIG. 3B and FIG. 1B, when the piston 3 rapidly moves downwards, an instantaneous high pressure pushes the multi-stage air pressure valve 1 so that the first component 11 is subjected to a greater downward force to compress the elastic piece 14, which makes the protrusion 112 being moved into the bottom part 121 of the second component 12. Since the gap between the protrusion part 112 and the bottom part 121 is sealed by the O-shaped ring, the second bypass channel 15 of the multi-stage air pressure valve 1 is blocked, so that the air that is compressed downwards can only be discharged from the first air outlet 1132 of the main channel 113.

In conclusion, when the piston 3 slowly reciprocates, the air in the lower air chamber 212 is discharged through the main channel 113, the bypass channel 114 and the bypass channel 115 of the multi-stage air pressure valve 1 (FIG. 1A) with a longer cushioning stroke so that the user can feel more comfortable. When the piston 3 rapidly reciprocates, the air in the lower air chamber 212 can only be discharged through the main channel 113 of the multi-stage air pressure valve 1 (FIG. 1B) with a shorter cushioning stroke so that the user is highly sensitive to the external environment (such as road surface).

### EMBODIMENT 4

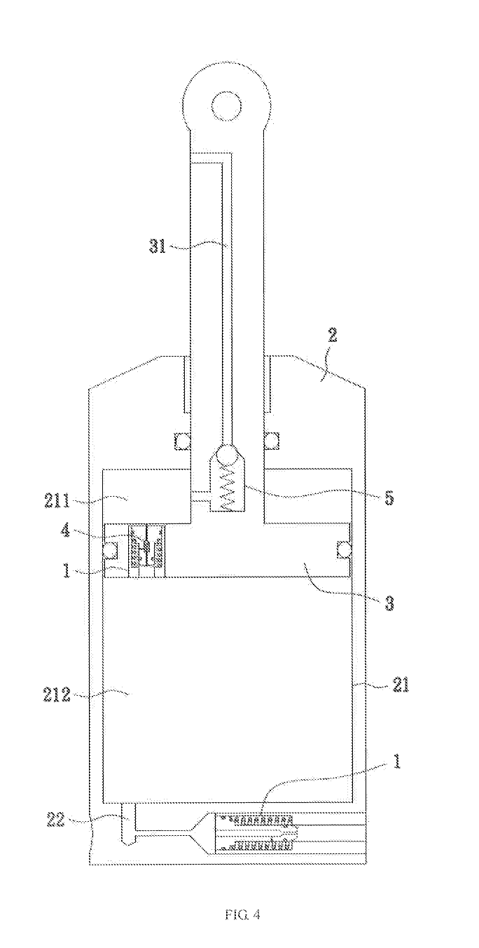

As shown in FIG. 4, the fourth embodiment is different from the third embodiment in that a multi-stage air pressure valve 1 is disposed inside the piston 3, and the first check valve 4 is disposed inside the multi-stage air pressure valve 1.

The airflow can be preliminarily adjusted when passing through the piston 3, and further adjusted for its gas flow rate via the multi-stage pressure valve 1 in the lower air way 22, so that the adjustment-free multi-stage prosthesis air cylinder having multiple stages is realized by the mutual-combination of the multi-stage air pressure valves I disposed in the piston 3 and in the lower air way 22.

### POTENTIAL EFFECTS OF THE EMBODIMENTS

The adjustment-free multi-stage prosthesis air cylinder of the present creation is suitable for prosthesis joint, mainly suitable for knee prosthesis for legs. When the users are slowly walking (usually with a walking speed less than 2-4 km/h), the gas flow rate is relatively larger, so as to quickly discharge the inside pressure, which enables a longer cushioning stroke for the piston so that the users can feel more comfortable. When the users are walking in a high speed (usually with a walking speed around 4-8 km/h), the gas flow rate of the multi-stage air pressure valve 1 is automatically reduced so that the inner thereof has an instantaneous high pressure, which enables a shorter cushioning stroke for the piston to provide the prosthesis with a good operability.

The adjustment-free multi-stage prosthesis air cylinder of the present creation is simple to install, so that the users do not need to adjust the setting of the pressure valve, but only need to assemble the adjustment-free multi-stage prosthesis air cylinder with the prosthesis joint, without requirements to check and adjust the pressure valve. Moreover, since the adjustment-free multi-stage prosthesis air cylinder has a simple structure, lower cost, and longer span lift, it overcomes the defects of the traditional installation that is complex, error-prone and requires frequent maintenance.

It should be stated that the above description only illustrates the preferred embodiments of the present creation and is not intended to limit the extent of scope thereof. Therefore all the equivalent changes by following the concepts of the specification and the drawings of the present creation should be fallen within the claimed extent of scope thereof.

What is claimed is:

1. A multi-stage air pressure valve disposed in the air way, comprising:
a first component, wherein the inner of the first component comprises a main channel and a first bypass channel, wherein the main channel penetrates through the first component, forms an air inlet on the top surface of the first component and forms a first air outlet on the bottom surface of the first component, and wherein the first bypass channel communicates with the main channel and forms a second air outlet on the side surface of the first component;
a second component, wherein the second component is optionally sleeved on the first component, and a second bypass channel communicating with the first bypass channel is formed between the first component and the second component;
two sealing rings, wherein the two sealing rings are disposed on the side surface of the first component to optionally close the second bypass channel, and the second air outlet is located between the two sealing rings; and
an elastic piece, the elastic piece is connecting the first component with the second component.

2. The multi-stage air pressure valve of claim 1, wherein the first component comprises a top part and a protrusion part, the second component comprises a bottom part and an extension part, wherein the protrusion part axially extends from the top part towards the second component, and the extension part axially extends from the bottom part towards the first component.

3. The multi-stage air pressure valve of claim 2, wherein one end of the elastic piece is connected to the top part and the other end of the elastic piece is connected to the bottom part, wherein the elastic piece surrounds the protrusion part.

4. The multi-stage air pressure valve of claim 2, wherein one of the two sealing rings is disposed on the top part, and the other one is disposed on the protrusion part.

5. The multi-stage air pressure valve of claim 1, wherein the first component forms into a tapered shape between the second outlet and the first outlet so as to form variable gaps between the first component and the second component.

6. An adjustment-free multi-stage prosthesis air cylinder applicable for a prosthesis joint, comprising:
an air cylinder body forming a lower air way, wherein an air chamber is formed inside the air cylinder body;
a piston, wherein one end of the piston is slidably disposed in the air chamber, and the other end of the piston extends out of the air cylinder body, wherein the piston divides the air chamber into an upper air chamber and a lower air chamber, wherein an upper air way is formed inside the piston, one end of the upper air way is connected to the outside and the other end of the upper air way is connected to the upper air chamber, wherein the lower air chamber communicates with the lower air way and wherein the piston makes a reciprocating movement in the air chamber;
a first check valve, wherein the first check valve is disposed in the piston to optionally connect the upper air chamber with the lower air chamber; and
a multi-stage air pressure valve disposed in the lower air way, wherein the multi-stage air pressure valve comprises: a first component, a second component, two sealing rings and an elastic piece, wherein the inner of the first component comprises a main channel and a first bypass channel, wherein the main channel penetrates through the first component, forms an air inlet on the top surface of the first component, and forms an first air outlet on the bottom surface of the first component and wherein the first bypass channel communicates with the main channel and forms a second air outlet on the side surface of the first component, wherein the second component is optionally sleeved on the first component, wherein a second bypass channel communicating with the first bypass channel is formed between the first component and the second component, wherein the two sealing rings are sleeved on the side surface of the first component to optionally close the second bypass channel, wherein the second air outlet is located between the two sealing rings, and wherein the elastic piece is connecting the first component with the second component.

7. The adjustment-free multi-stage prosthesis air cylinder of claim 6, further comprising a second check valve, wherein the second check valve is disposed in the upper air way so that the air can only flow from the outside of the air cylinder body to the upper air chamber in one-way.

8. The adjustment-free muiti-stage prosthesis air cylinder of claim 6, wherein the first component comprises a top part and a protrusion part, the second component comprises a bottom part and an extension part, wherein the protrusion part extends Out from the top part towards the second component, and the extension part extends out from the bottom part towards the first component.

9. The adjustment-free multi-stage prosthesis air cylinder of claim 8, wherein one end of the elastic piece is connected to the top part, and the other end of the elastic piece is connected to the bottom part; wherein the elastic piece surrounds the protrusion part.

10. The adjustment-free multi-stage prosthesis air cylinder of claim 6, wherein the multi-stage air pressure valve is provided with at least a second sealing ring to close the gap between the multi-stage air pressure valve and the lower air way.
